(19) United States
(12) Patent Application Publication
(10) Pub. No.: US 2007/0166837 A1
(43) Pub. Date: Jul. 19, 2007

(54) Nanoscale biomolecule sensor and method for operating same

(76) Inventors: Ying-Lan Chang, Cupertino, CA (US); Maozi Liu, Fremont, CA (US); Dan-Hui Dorothy Yang, Sunnyvale, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/334,981

(22) Filed: Jan. 19, 2006

(51) Int. Cl. G01N 33/543; C12M 3/00

(52) U.S. Cl. 436/518; 435/287.2; 977/902

(57) ABSTRACT

A nanoscale biomolecule sensor includes a nanoscale sensor element connected between a first electrical terminal and a second electrical terminal, the nanoscale sensor element coated with a capture agent. The sensor includes an electrode arrangement operable to establish a temporary electric field in the vicinity of the nanoscale sensor element, the temporary electric field oriented to move biomolecules of interest and other biomolecules having the same charge polarity as the biomolecules of interest toward the nanoscale sensor element where the biomolecules of interest specifically bind with the capture agent, the biomolecules of interest bound to the capture agent having an electric charge that changes an electrical property of the nanoscale sensor element measurable between the electrical terminals.

### BACKGROUND OF THE INVENTION

Micro-analytical sensors to detect extremely small concentrations of molecules in an analyte are currently being developed. These sensors are capable of detecting particular molecules in femtomolar (fM)-order concentrations, corresponding to a few thousand, or a few hundred, molecules in a sample volume of an analyte. These sensors are referred to as molecular, or biomolecular, sensors, and are being developed in nanometer (nm) scale proportions. For example, a biomolecular sensor employing a nanowire, nanotube, or other nanostructure-scale structure has been developed that can detect extremely small concentrations of DNA molecules in a sample volume. In one example in which the biomolecule sensor can be analogized to a field effect transistor (FET), a silicon nanowire doped with a dopant forms the channel of the FET. In the case of biomolecule detection, a biomolecule that carries an external charge functions as the gate, and is referred to as a “molecular gate.” The ends of the silicon nanowire have electrical connections that are connected to what can be described as the drain and source terminals of the FET. The drain and source terminals provide an electrical pathway so that the electrical properties (for example, voltage and current) of the silicon nanowire can be monitored and controlled.

In one example using an antibody and antigen as the biomolecules, the silicon nanowire is functionalized on its surface with an antibody with which a particular antigen will specifically bind. In this example, the antibody coats the surface of the silicon nanowire. In such an application, the silicon nanowire is referred to as a nanosensor element. An antibody is a protein used by the immune system to identify and neutralize foreign objects, such as bacteria and viruses. Each antibody recognizes a specific antigen and can form an antibody-antigen complex. The formation of the antibody-antigen complex or the specific binding between antibody and antigen on the surface of the silicon nanowire results in a change in the physical or chemical properties of the antibody. As an analogy, the charge on the gate of the nanosensor changes, thus the electrical properties of the nanowire FET are affected. Other molecules in which specific binding can occur, or in which a physical or chemical property can be changed due to the presence of a specific molecule, can also be used. These molecules that are used to functionalize the nanowire or nanotube are referred to as capture agents. Capture agents include, for example, proteins, peptides, and specific DNA or RNA sequences. The nanowire then functions as a biomolecule sensor.

The electrical properties of a nanowire are determined by the diameter of the nanowire and the doping applied to the nanowire. A protein, e.g. an antigen, has a net electrical charge that is related to its isoelectric point. The isoelectric point is a pH value at which the net electric charge of the protein is zero. However, as the pH value increases, the net charge of the protein becomes negative and as the pH value decreases the net charge of the protein becomes positive. Therefore, by monitoring and adjusting the pH value, the net electric charge of a biomolecule can be determined and controlled. A fluid containing the biomolecule to be analyzed is then directed toward the nanowire sensor. In one example, the nanowire sensor is located in a micro-fluidic channel and the fluid flows through the channel toward the nanowire sensor. If the fluid contains the particular biomolecule of interest, an antigen in this example, the antigen molecules will specifically bind with the antibodies which are present on the surface of the nanowire sensor. Because the antigens carry electric charge, when the antigens specifically bind to the antibodies on the nanowire sensor, the current flowing through the nanowire sensor is affected. If the electrical channel formed by the nanowire sensor is sufficiently small, a small amount of charge on the surface of the nanowire sensor will be sufficient to deplete the channel and cause a significant conductance change in the channel. By knowing the charge associated with a particular antigen (or other molecule) and by monitoring the current flowing through the nanowire sensor before and after the specific binding occurs, the presence of the antigen, and its concentration in the fluid can be determined.

Generally, scaling the above-described biomolecule sensor to nanometer-scale proportions increases the signal-to-noise ratio of the sensor, thereby improving the signal transduction and the sensitivity of the sensor. However, another consideration with respect to the sensitivity of the above-described biomolecule sensor relates to what is referred to as mass transport effect. Mass transport effect is related to the ability to direct the biomolecules in the fluid toward the sensor. Without the ability to direct the biomolecules in the fluid toward the sensor, a nanoscale sensor is generally limited to picomolar (pM)-order detection limits because of inefficient mass transport toward the nanoscale sensor.

### SUMMARY OF THE INVENTION

In an embodiment, a nanoscale biomolecule sensor comprises a nanoscale sensor element connected between a first electrical terminal and a second electrical terminal, the nanoscale sensor element coated with a capture agent. The sensor includes an electrode arrangement operable to establish a temporary electric field in the vicinity of the nanoscale sensor element, the temporary electric field oriented to move biomolecules of interest and other biomolecules having the same charge polarity as the biomolecules of interest toward the nanoscale sensor element. The biomolecules of interest specifically bind with the capture agent. The biomolecules of interest bound to the capture agent have an electric charge that changes an electrical property of the nanoscale sensor element measurable between the electrical terminals.

In another embodiment, the invention is a method for operating a nanoscale biomolecule sensor. The method comprises providing a nanoscale sensor element connected between a first electrical terminal and a second electrical terminal. The nanoscale sensor element is coated with a capture agent. The method also comprises temporarily establishing an electric field in the vicinity of the nanoscale sensor element. The temporary electric field is oriented to move biomolecules of interest and other biomolecules having the same charge polarity as the biomolecules of interest towards the nanoscale sensor element where the biomolecules of interest can specifically bind with the capture agent. The method also comprises measuring a change in an electrical property of the nanoscale sensor element, the change caused by electric charge carried by the biomolecules of interest specifically bound to the capture agent.

### BRIEF DESCRIPTION OF THE DRAWINGS

The invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram illustrating a biomolecule sensor implemented as a field effect transistor (FET).

FIG. 2 is a schematic diagram illustrating the nanowire sensor of FIG. 1.

FIGS. 3A through 3D are a series of schematic diagrams illustrating a nanoscale biomolecule sensor in accordance with an embodiment of the invention.

FIG. 4A is a schematic diagram illustrating a nanoscale biomolecule sensor constructed in accordance with another embodiment of the invention.

FIG. 4B is a cross-sectional view of the secondary structure of FIG. 4A.

FIG. 5 is a schematic diagram illustrating a nanoscale biomolecule sensor constructed in accordance with another embodiment of the invention.

FIG. 6A is a schematic diagram illustrating a nanoscale biomolecule sensor constructed in accordance with another embodiment of the invention.

FIG. 6B is a cross-sectional view of the secondary structure of FIG. 6A.

FIG. 7 is a flowchart illustrating a method of operating a nanoscale biomolecule sensor in accordance with an embodiment of the invention.

### DETAILED DESCRIPTION OF THE INVENTION

The nanoscale biomolecule sensor and method for operating same will be described below in the context of attracting an antigen to an antibody placed on the surface of a silicon nanowire biomolecule sensor element. However, other nanostructures can be implemented as the biomolecule sensor element. For example, a nanotube, or other nanostructure can be implemented as the biomolecule sensor element. Further, other biomolecules can be detected by placing the appropriate capture agents on the biomolecule sensor element. Further, while an antibody-antigen system is used as one example of a capture agent, other capture agents can be used.

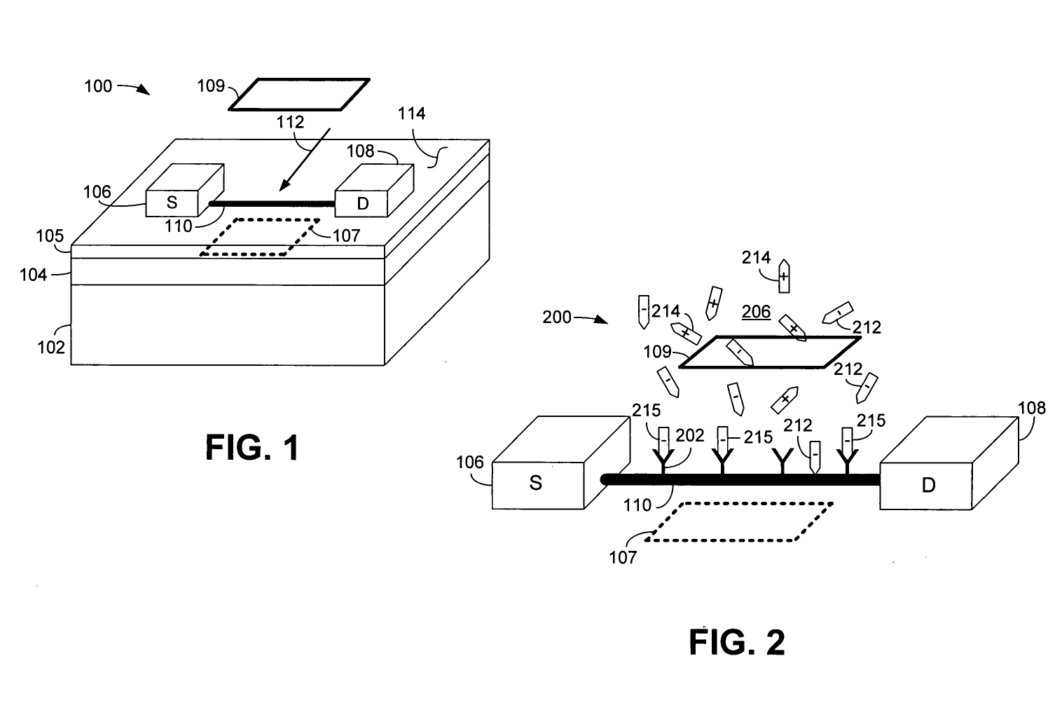

FIG. 1 is a schematic diagram illustrating a biomolecule sensor 100 implemented as a field effect transistor (FET). The biomolecule sensor 100 comprises a silicon substrate 102 over which a layer 104 of a dielectric is formed. The layer 104 can be, for example, silicon dioxide (SiO₂), or another dielectric. An electrode 107 is formed on a surface of the layer 104. Another dielectric is applied as a layer 105 over the electrode 107 and the layer 104. The layer 105 may be formed using, for example, silicon nitride (for example, Si₃N₄), or another dielectric. Another electrode 109 is located above the surface 114. The electrodes 107 and 109 may be referred to as an electrode arrangement. A source 106 and a drain 108 are formed on the layer 105. A nanowire biomolecule sensor element 110, hereafter referred to as sensor element 110, is formed on the surface of the layer 105 and is electrically connected to the source 106 and drain 108. In one embodiment, the sensor element 110 is formed of silicon and is doped p-type or n-type, depending on the biomolecule sought to be detected. The source 106 and drain 108 can be metallic contacts, such as gold. The sensor element 110 can be formed with a diameter of approximately 5 to 40 nanometers (nm) and with a length of approximately 2 micrometers (μm) using semiconductor fabrication techniques. The sensor element 110 rests on the surface 114 of the layer 105 or can be suspended above the surface 114 of the layer 105. The sensor element 110 is located between the electrodes 107 and 109 so that an electric field can be induced between the electrodes 107 and 109 and be applied in the vicinity of the sensor element 110 by a voltage applied to the electrodes 107 and 109, as will be described below. The arrow 112 indicates the direction of flow of fluid toward and past the sensor element 110. However, the flow direction shown is arbitrary. Further, a micro-fluidic channel (not shown) may be formed on the surface 114 of the layer 105 to direct the flow of fluid toward the sensor element 110. The sensor element 110 located between the source 106 and the drain 108 forms the channel of a field effect transistor.

FIG. 2 is a schematic diagram 200 illustrating the nanowire sensor 110 of FIG. 1. In the example shown in FIG. 2, the sensor element 110 is doped to make it electrically conductive and the surface of the sensor element 110 is functionalized with a capture agent 202 using techniques that are known in the art to make biomolecules specifically bind to it. A fluid containing an analyte is indicated using reference numeral 206 and is directed toward the sensor element 110. In an embodiment, the fluid 206 is a solution containing the analyte to be detected. However, the fluid 206 need not be a solution. The flow of the fluid can be directed toward the sensor element 110 using, for example, a micro-fluidic channel (not shown). The micro-fluidic channel through which the fluid 206 flows can be of the order of several micrometers (μm) in width and depth. The fluid 206 moves toward the sensor element 110 due to both flow as described above and due to the application of an electric field between the electrodes 107 and 109, as will be described below. The fluid contains a variety of biomolecules, some having a positive electrical charge and some having a negative electrical charge. The biomolecules having negative electrical charge are generally illustrated using reference numeral 212 and the biomolecules having positive electrical charge are generally illustrated using reference numeral 214. In this example, the biomolecules 212 and 214 are antigens and the capture agent 202 is an antibody to which particular antigens will bind.

The fluid 206 may contain a number of different positively-charged and negatively-charged biomolecules. However, only particular biomolecules will specifically bind to the capture agent 202. These biomolecules are shown as specifically-bound to the capture agent 202 using reference numeral 215. However, other negatively-charged biomolecules 212 will be attracted to the surface of the sensor element 110, and will influence the electrical properties of the sensor element 110, thus causing errors when attempting to detect the specifically-bound biomolecules. In this example, the capture agent 202 may comprise biomolecules, such as antibodies, proteins, peptides, DNA or RNA sequences. In this example, the biomolecules of interest are chosen from an antigen, donor, protein, peptide, receptor, ligand and a nucleotide. However, other capture agents and biomolecules may be used.

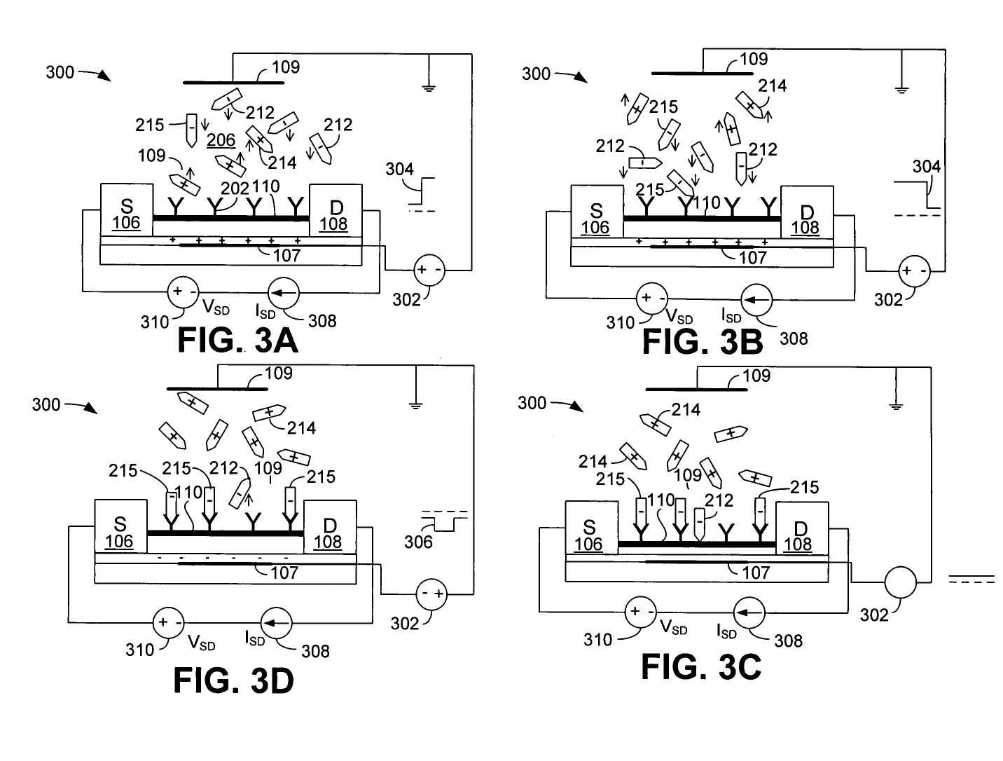

FIGS. 3A through 3D are a series of a schematic diagrams illustrating a nanoscale biomolecule sensor in accordance with an embodiment of the invention. FIG. 3A is a schematic diagram illustrating a nanoscale biomolecule sensor 300. The nanoscale biomolecule sensor 300 includes a sensor element 110 which has been functionalized with a capture agent 202, in this example an antibody, as discussed above. The fluid 206 comprises negatively-charged biomolecules 212 and positively-charged biomolecules 214. In this example, the biomolecules 212 and 214 are antigens. In this example, a variety of different positively-charged and negatively-charged biomolecules are present in the fluid 206. A voltage source 302 is connected to the electrodes 107 and 109 to enable the application of an electrical voltage that creates a temporary electric field in the fluid 206 in the vicinity of the sensor element 110. A monitor voltage source 310 and a current monitor 308 are connected in series between the source 106 and the drain 108 to allow the electrical properties of the sensor element 110 to be monitored. The voltage source 302, monitor voltage source 310 and current monitor 308 are examples of the circuitry that can be used to create a temporary electric field and monitor the electrical properties of the sensor element 110. Other circuitry may be used.

In accordance with an embodiment of the invention, the voltage source 302 applies an electrical pulse 304 between the electrodes 107 and 109. This creates a temporary electric field in the fluid 206 in the vicinity of the sensor element 110. In the example shown here, the electrical pulse 304 is a positive electrical pulse to attract negatively-charged biomolecules 212 and 215 to the sensor element 110. To attract positively-charged biomolecules 214 to the sensor element 110, the electrical pulse 304 would have negative polarity. The magnitude and duration of the electrical pulse 304 can be determined based on the characteristics of the fluid and the particular biomolecule sought to be attracted. For example, depending on the application and the design of the sensor element 110, a single pulse or a pulse train may be applied to the sensor element 110. An exemplary voltage range of 100 millivolts (mV) to several volts (V), and a pulse width of approximately 10 milliseconds (ms) to 1 second (s) are possible. However, other voltages and pulse widths may be used.

FIG. 3B is a schematic diagram illustrating the nanoscale biomolecule sensor 300 and the sensor element 110 during the application of the electrical pulse 304. The motive force applied to the negatively-charged biomolecules by the electric field causes the negatively-charged biomolecules 212 and 215 in the fluid 206 to migrate toward the sensor element 110. The motive force applied to the positively-charged biomolecules causes them to migrate away from the sensor element 110. The electric field alters the mass transport characteristics of the biomolecules in the fluid 206 so that the biomolecules having one electric charge polarity are drawn towards the sensor element 110 and those having the opposite polarity are moved away from the sensor element 110. The application of the electrical pulse 304 causes the biomolecules having a negative electric charge polarity to move toward the sensor element 110. The movement of the biomolecules having a negative electric charge polarity toward the sensor element 110 increases the local concentration of such biomolecules within a distance on the order of nanometers (nm) from the sensor element 110, thus greatly enhancing the probability of specific binding between the biomolecules and the capture agent 202. However, the electric field attracts all negatively-charged biomolecules 212 toward the sensor element 110.

FIG. 3C is a schematic diagram illustrating the nanoscale biomolecule sensor 300 and the sensor element 110 after the application of the first electrical pulse 304. The fluid 206 typically contains a number of different negatively-charged biomolecules. However, only particular negatively-charged biomolecules, referred to as the biomolecules of interest, will specifically bind to the capture agent 202. These biomolecules are shown as specifically-bound to the capture agent 202 using reference numeral 215. However, due to the positive electric charge imparted to the sensor element 110, other negatively-charged biomolecules 212 will also be attracted to the sensor element 110. The electrical charge associated with these biomolecules 212 will influence the electrical properties of the sensor element 110, thus causing errors when attempting to detect the change in electrical properties of the sensor element 110 due to the specifically-bound biomolecules 215.

In accordance with an embodiment of the invention, and as shown in FIG. 3D, a second electrical pulse 306 having a polarity opposite the polarity of the electrical pulse 304 is applied to the electrodes 107 and 109 as described above. In this example, the electrical pulse 306 has a negative polarity, and is generally smaller in magnitude than the electrical pulse 304, but the magnitude may be equal to or greater than the magnitude of the electrical pulse 304. The temporary electric field resulting from applying the electrical pulse 306 between the electrodes 107 and 109 causes the non-specifically-bound biomolecules 212 to move away from the sensor element 110. Because the interaction between the capture agent 202 on the sensor element 110 and the biomolecule 212 is much weaker than the specific binding between the capture agent 202 and the specifically-bound biomolecules of interest (biomolecules of interest 215), the specifically-bound biomolecules 215 are not repelled and remain bound to the capture agent 202. In this manner, after the application of the electrical pulse 306, only the biomolecules 215 that are specifically bound to the capture agent 202 affect the electrical properties of the sensor element 110. By monitoring the current driven through the sensor element 110 by the monitor voltage source 310 before and after the specific binding of the biomolecules 215 using the current monitor 308, it can be determined whether the biomolecules 215 (i.e., the biomolecules of interest) are present in the fluid 206. Further, because the application of the electrical pulse 304 causes the biomolecules of interest 215 to rapidly approach and specifically bind with the capture agent 202 (e.g. an antibody), and because the subsequent application of the electrical pulse 306 repels the non-specifically binding biomolecules 212 from the sensor element 110, a very small concentration of biomolecules of interest 215 in the fluid 206 can be detected. For example, concentrations of biomolecules 215 in the femtomolar range can be detected by the biomolecule sensor 300. This enables the sensor element 110 to be highly selective and highly sensitive with a fast response time. The biomolecules of interest 215 that are specifically-bound to the sensor element 110 act as a “molecular gate” and change the conductance of the sensor element 110.

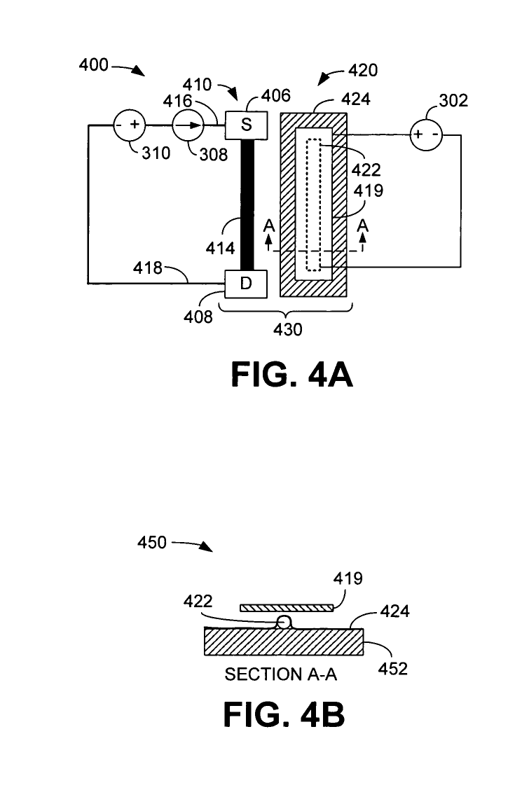

FIG. 4A is a schematic diagram illustrating a nanoscale biomolecule sensor 400 constructed in accordance with another embodiment of the invention. The nanoscale biomolecule sensor 400 comprises a biomolecule sensor portion 410 and a secondary structure 420. The biomolecule sensor portion 410 comprises a nanowire, or nanotube, 414 that is similar to the sensor element 110 described above. A monitor voltage source 310 and a current monitor 308 are connected in series between source terminal 406 and the drain terminal 408 of the sensor portion 410 via connections 416 and 418. The secondary structure 420 comprises a nanostructure 422 that is covered by a dielectric 424, such as silicon nitride (SiNₓ) in the case of a silicon nanowire FET sensor element, to prevent binding of biomolecules to the nanostructure 422. However, the dielectric material is chosen based on the materials used to fabricate the secondary structure 420. An electrode 419, which is similar to the electrode 109 described above, is located over the nanostructure 422 to subject the secondary structure 420 to an electric field when a voltage is applied between the nanostructure 422 and the electrode 419. The nanowire 414, the nanostructure 422 and the electrode 419 comprise a sensor element 430.

In this embodiment, the biomolecule sensor portion 410 is used as the sensor to detect the presence of a particular biomolecule and the secondary structure 420 is used to attract the biomolecules of interest toward the surface of the nanowire 414. The voltage source 302 and the monitor voltage source 310 are independently controlled so that the current flowing through the nanowire 414 is not interrupted when the voltage pulse described above is applied in the vicinity of the sensor element 430. Instead of being applied to electrodes associated with the nanowire 414, the voltage pulse is applied between the nanostructure 422 and the electrode 419.

The secondary structure 420 is placed in sufficiently close proximity to the biomolecule sensor portion 410 so that when the biomolecules of interest are attracted to the nanostructure 422 as described above, the biomolecules of interest specifically bind to the nanowire 414. By bringing the biomolecules of interest sufficiently close to the surface of the nanowire 414, specific binding may occur between the biomolecules of interest and the capture agent 202 (not shown) on the surface of the nanowire 414. In one example using current processing technology, the nanowire 414 and the nanostructure 422 are separated by a distance on the order of approximately 200 nm to a few micrometers (μm), and may be separated by approximately as much as four micrometers. The proximity of the nanostructure 422 to the nanowire 414 allows an increase in the local concentration of biomolecules of interest near the nanowire 414, and therefore increases the sensitivity and selectivity of specific binding between the biomolecules of interest and the antibodies on the nanowire 414.

FIG. 4B is a cross-sectional view of the secondary structure of FIG. 4A through section A-A. The nanostructure 422 is formed over a substrate 452 as described above. In FIG. 4B, the nanostructure 422 is shown as being in contact with the substrate 452; however, the nanostructure 422 need not be in contact with the substrate 452. A dielectric 424, such as silicon nitride (SiNₓ), is applied as a film over the nanostructure 422 to prevent binding of biomolecules to the nanostructure 422. The dielectric material is chosen based on the materials used to fabricate the secondary structure 420. The electrode 419 is located over the nanostructure 422 to create an electric field in the vicinity of the sensor element 430 when a voltage is applied between the nanostructure 422 and the electrode 419.

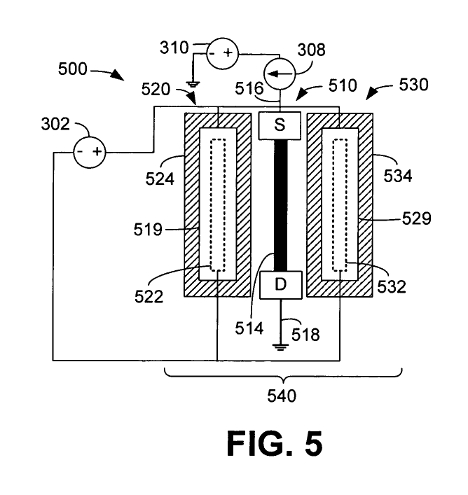

FIG. 5 is a schematic diagram illustrating a nanoscale biomolecule sensor 500 constructed in accordance with another embodiment of the invention. The nanoscale biomolecule sensor 500 comprises a biomolecule sensor portion 510 and two secondary structures 520 and 530. The biomolecule sensor portion 510 comprises a nanowire 514 that is similar to the sensor element 110 described above. The nanowire 514 is connected in series with a monitor voltage source 310 and a current monitor 308 via connection 516 and is coupled to ground via connection 518.

The secondary structure 520 comprises a nanostructure 522 that is covered by a dielectric 524 to prevent binding of biomolecules to the nanostructure 522. The dielectric 524 is similar to the dielectric 424, described above. An electrode 519 is located over the nanostructure 522. The electrode 519 is connected to one output of the voltage source 302. The nanostructure 522 is connected to the other output of the voltage source 302. The secondary structure 530 comprises a nanostructure 532 that is covered by a dielectric 534, which is similar to the dielectric 524 to prevent binding of biomolecules to the nanostructure 532. An electrode 529 is located over the nanostructure 532. The electrode 529 is connected to one output of the voltage source 302. The nanostructure 532 is connected to the other output of the voltage source 302. The nanowire 514, the nanostructure 522, the electrode 519, the nanostructure 532 and the electrode 529 comprise a sensor element 540. In this embodiment, the biomolecule sensor portion 510 is used as the sensor to detect the presence of a particular biomolecule and the secondary structures 520 and 530 are used to attract the desired biomolecules toward the surface of the sensor element 514.

The secondary structures 520 and 530 are placed in sufficiently close proximity to the biomolecule sensor portion 510 so that when the biomolecules of interest are attracted to the nanostructures 522 and 532, as described above, the biomolecules of interest specifically bind to the nanowire 514. By bringing the biomolecules of interest sufficiently close to the surface of the nanowire 514, specific binding may occur between the biomolecules of interest and the capture agent 202 (not shown) on the surface of the nanowire 514. In one example using current processing technology, the nanowire 514 and the nanostructures 522 and 532 are separated by a distance on the order of approximately 200 nanometers (nm) to a few micrometers (μm) and may be separated by approximately as much as four micrometers. The proximity of the nanostructures 522 and 532 to the nanowire 514 allows an increase in the local concentration of biomolecules of interest near the nanowire 514, and therefore increases the sensitivity and selectivity of specific binding between the biomolecules of interest and the antibodies (not shown) on the nanowire 514.

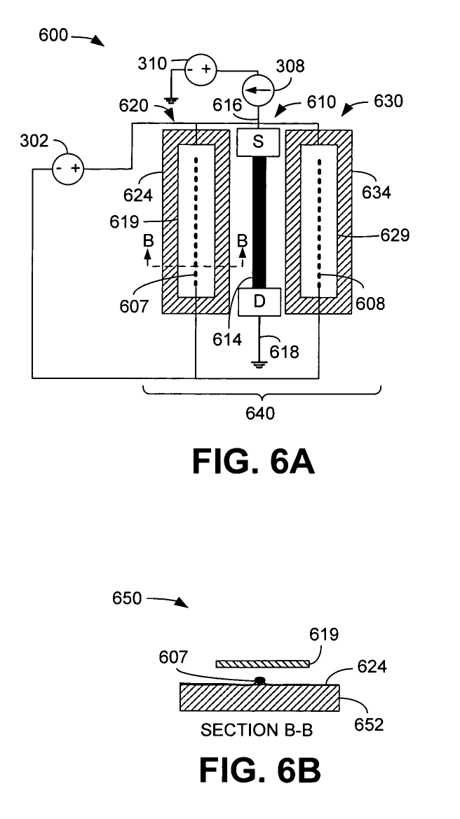

FIG. 6A is a schematic diagram illustrating a nanoscale biomolecule sensor 600 constructed in accordance with another embodiment of the invention. The nanoscale biomolecule sensor 600 is similar to the nanoscale biomolecule sensor 500 except that the nanostructures 522 and 532 are replaced by electrical conductors 607 and 608. The nanoscale biomolecule sensor 600 comprises a biomolecule sensor portion 610 and two secondary structures 620 and 630. The biomolecule sensor portion 610 comprises a nanowire 614 that is similar to the sensor element 110 described above. The nanowire 614 is connected in series with a monitor voltage source 310 and a current monitor 308 via connection 616 and is coupled to ground via connection 618.

The secondary structure 620 comprises an electrode 607 that is covered by a dielectric 624 to prevent binding of biomolecules to the electrode 607. The dielectric 624 is similar to the dielectric 424, described above. An electrode 619 is located over the electrode 607. The electrode 619 is connected to one output of the voltage source 302. The electrode 607 is connected to the other output of the voltage source 302. The secondary structure 630 comprises an electrode 608 that is covered by a dielectric 634, which is similar to the dielectric 624 to prevent binding of biomolecules to the electrode 608. An electrode 629 is located over the electrode 608. The electrode 629 is connected to one output of the voltage source 302. The electrode 608 is connected to the other output of the voltage source 302. The nanowire 614, the electrodes 607, 619, 608 and 629 comprise a sensor element 640. In this embodiment, the biomolecule sensor portion 610 is used as the sensor to detect the presence of a particular biomolecule and the secondary structures 620 and 630 are used to attract the desired biomolecules toward the surface of the sensor element 614.

The secondary structures 620 and 630 are placed in sufficiently close proximity to the biomolecule sensor portion 610 so that when the biomolecules of interest are attracted to the secondary structures 620 and 630 as described above, the biomolecules of interest specifically bind to the nanowire 614. By bringing the biomolecules of interest sufficiently close to the surface of the nanowire 614, specific binding may occur between the biomolecules of interest and the capture agent 202 (not shown) on the surface of the nanowire 614. In one example using current processing technology, the nanowire 614 and the electrodes 607, 619, 608 and 629 are separated by a distance on the order of approximately 200 nm to a few micrometers (μm) and may be separated by approximately as much as four micrometers. The proximity of the electrodes 607, 619, 608 and 629 to the nanowire 614 allows an increase in the local concentration of biomolecules of interest near the nanowire 614, and therefore increases the sensitivity and selectivity of specific binding between the desired biomolecules and the capture agent (not shown) on the nanowire 614.

FIG. 6B is a cross-sectional view of the secondary structure of FIG. 6A through section B-B. The electrode 607 is formed over a substrate 652 as described above. A dielectric 624, such as silicon nitride (SiNₓ), is applied as a film over the electrode 607 to prevent binding of biomolecules to the electrode 607. The dielectric material is chosen based on the materials used to fabricate the secondary structure 620. The electrode 619 is located over the electrode 607 to create an electric field in the vicinity of the sensor element 640 when a voltage is applied between the electrode 607 and the electrode 619.

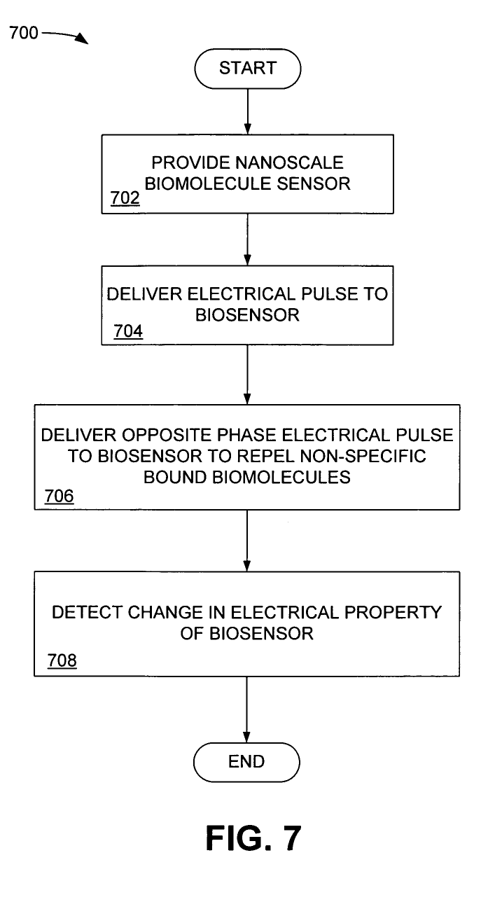

FIG. 7 is a flowchart 700 illustrating a method of operating a nanoscale biomolecule sensor in accordance with an embodiment of the invention. In block 702, a nanoscale biomolecule sensor element is provided. The surface of the nanoscale biomolecule sensor element is coated or otherwise functionalized with a capture agent comprising biomolecules, such as the antibodies, proteins, peptides, DNA or RNA sequences described above. In block 704, an electrical pulse is delivered to electrodes associated with the nanoscale biomolecule sensor element. The electrical pulse creates a temporary electric field between the electrodes so that biomolecules in the fluid in the electric field experience a motive force. The motive force causes biomolecules having a charge that is opposite the charge in the electric field to be attracted to the sensor element. Biomolecules that specifically bind with the capture agent on the sensor element as well as biomolecules that will not specifically bind with the capture agent on the sensor element are attracted to the sensor element. In block 706, an electrical pulse having a polarity opposite the polarity of the first electrical pulse is optionally delivered to the electrodes associated with the sensor element. The electrical pulse having a polarity opposite the polarity of the first electrical pulse creates a second temporary electric field between the electrodes so that biomolecules in the fluid in the electric field experience a motive force. The motive force repels away from the sensor element the biomolecules having the same charge polarity as the biomolecules of interest, but that do not specifically bind with the capture agent on the sensor element. In block 708, a change in the electrical properties of the sensor element is measured to detect the presence and the concentration of the specifically-bound biomolecules.

This disclosure describes the invention in detail using illustrative embodiments. However, it is to be understood that the invention defined by the appended claims is not limited to the precise embodiments described.

What is claimed is:

1. A nanoscale biomolecule sensor, comprising:
a nanoscale sensor element connected between a first electrical terminal and a second electrical terminal, the nanoscale sensor element coated with a capture agent; and
an electrode arrangement operable to establish a temporary electric field in the vicinity of the nanoscale sensor element, the temporary electric field oriented to move biomolecules of interest and other biomolecules having the same charge polarity as the biomolecules of interest toward the nanoscale sensor element where the biomolecules of interest specifically bind with the capture agent, the biomolecules of interest bound to the capture agent having an electric charge that changes an electrical property of the nanoscale sensor element measurable between the electrical terminals.

2. The nanoscale biomolecule sensor of claim 1, in which:
the temporary electric field is a first temporary electric field and has a first direction; and
the electrode arrangement is additionally operable to establish a second temporary electric field temporally following the first temporary electric field, the second temporary electric field oriented to move the other biomolecules having the same charge polarity as the biomolecules of interest but not bound to the capture agent away from the nanoscale sensor element.

3. The nanoscale biomolecule sensor of claim 2, in which the nanoscale sensor element comprises a nanowire.

4. The nanoscale biomolecule sensor of claim 3, in which the nanowire constitutes the channel of a field effect transistor.

5. The nanoscale biomolecule sensor of claim 4, in which the electric charge associated with the biomolecules of interest alters electric current flowing through the nanowire between the first and second terminals.

6. The nanoscale biomolecule sensor of claim 2, in which the nanoscale sensor element comprises a nanotube.

7. The nanoscale biomolecule sensor of claim 2, in which the biomolecules of interest are chosen from an antigen, donor, protein, peptide, receptor, ligand and a nucleotide.

8. The nanoscale biomolecule sensor of claim 2, in which the least detectable concentration of the biomolecules of interest is on the order of one picomole.

9. The nanoscale biomolecule sensor of claim 1, additionally comprising an additional nanostructure located proximate to the nanoscale sensor element, wherein the additional nanostructure constitutes part of the electrode arrangement.

10. The nanoscale biomolecule sensor of claim 9, in which:
the temporary electric field is a first temporary electric field and has a first direction; and
the electrode arrangement comprising the additional nanostructure is additionally operable to generate a second temporary electric field temporally following the first temporary electric field, the second temporary electric field oriented to move the other biomolecules having the same charge polarity as the biomolecules of interest but not bound to the capture agent away from the nanoscale sensor element.

11. The nanoscale biomolecule sensor of claim 9, in which the nanoscale sensor element and the additional nanostructure are separated by a distance in the range from approximately 200 nanometers to approximately four micrometers.

12. The nanoscale biomolecule sensor of claim 9, in which the additional nanostructure comprises one of a nanowire, a nanotube and an electrical conductor.

13. The nanoscale biomolecule sensor of claim 9, in which the biomolecules of interest are chosen from an antigen, protein, peptide, receptor, ligand, donor, and a nucleotide.

14. The nanoscale biomolecule sensor of claim 9, in which the nanoscale sensor element constitutes the channel of a field effect transistor.

15. The nanoscale biomolecule sensor of claim 14, in which the electric charge associated with the biomolecules of interest alters electric current flowing through the nanowire between the first and second terminals.

16. The nanoscale biomolecule sensor of claim 9, in which the least detectable concentration of the biomolecules of interest is on the order of one picomole.

17. A method for operating a nanoscale biomolecule sensor, the method comprising:
providing a nanoscale sensor element connected between a first electrical terminal and a second electrical terminal, the nanoscale sensor element coated with a capture agent;
in the vicinity of the nanoscale sensor element, temporarily establishing an electric field oriented to move biomolecules of interest and other biomolecules having the same charge polarity as the biomolecules of interest towards the nanoscale sensor element where the biomolecules of interest can specifically bind with the capture agent; and
at the electrical terminals, measuring a change in an electrical property of the nanoscale sensor element, the change caused by electric charge carried by the biomolecules of interest specifically bound to the capture agent.

18. The method of claim 17, in which:
the electric field is a first electric field; and
the method additionally comprises temporarily establishing a second electric field in the vicinity of the nanoscale sensor element, the second electric field oriented to move the other biomolecules not specifically bound to the capture agent away from the nanoscale sensor element.

19. The method of claim 18, in which:
the method additionally comprises providing an additional nanostructure and an electrode; and
the establishing comprises applying a voltage between the additional nanostructure and the electrode.

20. A nanoscale biomolecule sensor, comprising:
a nanoscale sensor element connected between a first electrical terminal and a second electrical terminal, the nanoscale sensor element coated with a capture agent;
an electrode arrangement located in the vicinity of the nanoscale sensor element, a first electrical pulse applied to the electrode arrangement establishing a first electric field that moves biomolecules of interest and other biomolecules having the same charge polarity as the biomolecules of interest towards the nanoscale sensor element where the biomolecules of interest can specifically bind with the capture agent, the biomolecules of interest bound to the capture agent having an electric charge that changes an electrical property of the nanoscale sensor element measurable between the electrical terminals; and
a second electrical pulse opposite in polarity to the first electrical pulse applied to the electrode arrangement establishing a second electric field that moves the other biomolecules not specifically bound to the capture agent away from the nanoscale sensor element.
